(19) United States
(12) Patent Application Publication
(10) Pub. No.: US 2004/0156816 A1
(43) Pub. Date: Aug. 12, 2004

(54) Lipid-drug complexes in reversed liquid and liquid crystalline phases

(76) Inventor: David Anderson, Ashland, VA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/635,019

(22) Filed: Aug. 6, 2003

Related U.S. Application Data
(60) Provisional application No. 60/401,011, filed on Aug. 6, 2002.

(51) Int. Cl. A61K 7/075; A61K 7/08

(52) U.S. Cl. 424/70.22; 424/70.27

(57) ABSTRACT

A pharmaceutical is formulated to enable enhanced delivery across membrane barriers, permit solubilization, protect compounds from deactivation by thiol containing compounds in the body, and allow retention of the drug during transport to a desired site of activity. The pharmaceutical includes a complex of two moieties where at least one is pharmaceutically active and is larger than a single atom in size, and the second moiety, when combined with a cationic or anionic counterion forms either a pharmaceutically acceptable anionic or cationic surfactant or a pharmaceutically acceptable salt that has an octanol water partition coefficient of greater than about 100.

This application claims priority to U.S. Provisional Patent Application 60/401,011 filed Aug. 6, 2002.

### BACKGROUND OF THE INVENTION

1. Field of the Invention

The invention is directed to drug formulation techniques which enable enhanced delivery of drugs or other pharmaceutically actives across membrane barriers, permit solubilization, protect compounds from deactivation by thiol containing compounds in the body, and allow retention of the drug during transport to a desired site of activity.

2. Description of the Related Art

Lipid-based materials, particularly microparticles, are an attractive alternative for the delivery of pharmaceutical actives, such as anticancer drugs in particular and especially platinum-based anticancer compounds which are currently the most widely used anticancer therapeutics. Interestingly, platinum-based anticancer compounds are the most amenable to improvement through advanced drug-delivery means. Lipid- and surfactant-based materials include such vehicles as liposomes, micelles, cochleates, and particles based on lyotropic liquid crystals such as lamellar phases, hexagonal phases, and cubic phases. The lipidic basis of these materials carry inherent advantages, such as biocompatibility, low toxicity, biodegradability, and, for some such materials, the potential for unique interactions with biomembranes that can be utilized to achieve efficient cell uptake, targeting to specific cells or organs, and even intracellular targeting, for example to the nucleus or mitochondria. Since platinum compounds are currently the most important class of drugs in the treatment of cancer, optimization of delivery vehicles for these compounds is of high importance.

However, in order to achieve these goals, several challenges must be met which are not adequately addressed by current approaches in the delivery of anticancer drugs, particularly as exemplified by platinum anticancer compounds:

1) First, the compound should be solubilized in the vehicle, because drugs administered in solid form typically exhibit low cellular uptake and can pose serious and immediate threats, such as the risk of pulmonary emboli. However, many of the most important platinum drugs are of low solubility in both water and typical lipids, or phrased more succinctly, they are of low solubility in typical lipid-water systems.

2) Second, even when solubilization in a lipid-water system is accomplished, encapsulation efficiency and retention of drug in the particle during transport to the tumor site should be as high as possible. In current systems, these can be quite low.

3) Third, the vehicle should have interactions with biomembranes that favor delivery of the drug to the cell. However, liposomes in particular are not pre-disposed to fusing with cell plasma membranes, and when they enter the cell via endocytosis they can become immobilized in endosomes.

4) Fourth, the ideal vehicle should protect drug compounds from detrimental binding and/or deactivation by proteins, e.g., for platinum drugs in particular, from deactivation by thiol-containing compounds in the body, particularly glutathione and albumin. This is a difficult task for a vehicle that needs to be labile enough to transfer the drug to biomembranes in a facile manner.

The ultimate delivery vehicle would solve these four challenges simultaneously, preferably within the context of a lipid-based delivery system with its associated biocompatibility.

Burger et al. [Nature Medicine 8, 81-84] describe a system in which acidic lipids are used to encapsulate cisplatin. The cisplatin is not solubilized in the lipid-water system. Rather, it is dispersed. Thus, their approach does not satisfy the first requirement given above. Furthermore, there does not seem to be any indication that the third requirement, of promoting fusion with membrane absorption barriers, is met by the vehicle.

The term liposome is frequently interchanged with the term vesicle and is usually reserved for vesicles of glycerophospholipids or other natural lipids. Vesicles are self-supported closed bilayer assemblies of several thousand lipid molecules (amphiphiles) that enclose an aqueous interior volume. The lipid bilayer is a two-dimensional fluid composed of lipids with their hydrophilic head groups exposed to the aqueous solution and their hydrophobic tails aggregated to exclude water. The bilayer structure is highly ordered yet dynamic because of the rapid lateral motion of the lipids within the plane of each half of the bilayer. See O'Brien. D. F. and Rarnaswami, V. (1989) in Mark-Bikales-Overberger-Menge Encyclopedia of Polymer Science and Engineering, Vol. 17, Ed. John Wiley & Inc., p. 108. Liposomes exhibit a number of limitations. Among these are their physical and chemical instabilities. The release of a material disposed within the liposome is usually dependent on the destabilization of the structure of the liposome. In particular, the absence of porosity precludes the pore-controlled release of such materials. The dual requirements of 1) physical stability of the liposome until release is desired on the one hand and 2) release of materials by bilayer destabilization when release is desired on the other, are problematic. Lamellar liquid crystalline phases, when dispersed in water, have a strong tendency to form closed, nonporous structures such as liposomes due to the high free energy cost of direct contact between water and the edges of lamellae.

Furthermore, as a necessary requirement for shelf stability, liposomes broadly exhibit limited tendency to interact strongly with lamellar bilayer systems, and in particular with biomembranes. The low, or zero, mean curvature of the bilayer midplane in lamellar and liposomal systems, and absence (or at least relative absence) of porosity, correlate with this lack of fusion with biomembranes.

Lynch and Spicer (U.S. patent application 2002/0153509) describe cubic phase gels based on the monoglyceride, monoolein, and di(canola ethyl ester) dimethylamine chloride (DEEDAC), dioctylamine HCl (DOAC*HCl), or dioctadecyl dimethyl ammonium chloride (DODMAC), and the drug ketoprofen, and demonstrate modified release of the drug from the cubic phase. However, Lynch and Spicer simply mix an anionic drug into a composition containing a cationic surfactant, and do not disclose a method for achieving a high degree of binding between a drug and a surfactant in a cubic phase or other phase, viz., so as to prevent release of the drug from the matrix. In their compositions, any binding between drug and surfactant (or “anchor”, in their terminology) is transient, and does not effectively bind the drug inside the liquid crystal, because counterions that are present (e.g., chloride) from the surfactant easily displace the drug. Thus, for example, in the dispersions reported in that disclosure, the ketoprofen is not effectively bound inside the particles by virtue of any electrostatic interaction with the surfactant (notwithstanding the fact that it may partition preferentially in the particles due to a hydrophobic interaction with the hydrophobic chains of the surfactant, as opposed to any interaction with the ionic polar head group). This is evidenced by the leakage of drug out of the particles into water, as reported in the patent of Lynch and Spicer. Furthermore, monoolein is extremely toxic when injected, and neither DEEDAC, DOAC, nor DODMAC are acceptable even for oral drug delivery, much less parenteral.

The approach, as typified by carboplatin, of synthesizing diammonium platinum compounds with very low-MW, water-soluble acids (such as oxalic acid, or cyclobutane dicarboxylic acid) coordinated to the platinum instead of chlorides is not a solution to the problem. The purpose of this approach has been to yield complexes with much higher water solubility than cisplatin, and thus would have a high tendency to diffuse out of and away from porous nanostructured phases such as reversed cubic, reversed hexagonal, and L3 phases, and thus this approach teaches away from solutions to the four-part challenge which was described above.

### SUMMARY OF THE INVENTION

Several mathematical analyses of the relationship between curvature properties, porosity, and fusion tendencies have been published. See, for example, Anderson, D. M., Wennerstrom, H. and Olsson, U. (1989) J. Phys. Chem. 93:4532. To summarize a crucial aspect of this, if one assumes a mathematical model in which the bilayer thickness is constant, and that the bilayer midplane is twice differentiable, one can show first that, in order to minimize unfavorable curvature energies, the midplane must have zero mean curvature throughout. Next, under these conditions one can then show that if the average mean curvature at the polar-apolar interface is toward water—as it is in a reversed liquid crystalline phase—then the integral Gaussian curvature is significantly negative. Negative integral Gaussian curvature then implies porosity in the bilayer system. A conclusion of the full analysis is that, if a composition which assembles into a porous bilayer phase, such as a reversed cubic phase, begins to exchange material with a membrane, such as a biomembrane, it can induce a local tendency for reversed curvature (curvature toward water at the polar-apolar interface), and thereby induce porosity in the biomembrane. This can be of great importance in the delivery of drugs across biomembrane barriers to absorption, constituting an inherent advantage of a reversed cubic or reversed hexagonal phase over a lamellar or liposomal material in the practice of drug delivery, particularly in the delivery of anticancer drugs where absorption barriers are very significant problems in therapeutic treatment. This is particularly true in the case of platinum drugs, which act directly on DNA and thus must penetrate deep into the target cell.

While the porous nanostructured phases, namely the reversed cubic, reversed hexagonal, and L3 phases, have this advantage of exhibiting interactions with biomembranes that favor delivery of the drug to the cell, they have the disadvantage that their porosity provides the opportunity for drugs to escape prematurely. That is, before the drug matrix reaches the site that is optimal, from the therapeutic point of view, for the release of the drug (such as at a tumor site, or metastatic site, or just at the surface of the intestinal epithelium or other absorptive tissue). Means have been described for coating these reversed phase materials, so as to prevent this premature release, and ultimately to allow targeting and other sophisticated approaches. The author has reported such methods in U.S. Pat. No. 6,482,517 which is hereby incorporated by reference. However, even in many of these processes, the pharmaceutical active must be substantially retained inside the porous, nanostructured material at crucial periods when the coating is not intact: in particular, during certain steps during the encapsulation process, and after dissolution or other release of the coating commences and it is still desirable to retain the drug. An especially important example of the latter is in the case where strong interactions between the porous matrix and a biomembrane barrier are anticipated, and a strong association between the drug and the matrix would carry the drug deep into the biomembrane, or even across it. While partitioning of the drug into the matrix, by virtue of a hydrophobic interaction, can provide an association of this sort for some drugs, for other drugs which have a lower partition coefficient, it typically cannot. Charged drugs are, of course, much more commonly substantially hydrophilic and typically exhibit lower partition coefficients.

Realizing the full potential of lipid systems for the encapsulation and delivery of drugs across membrane barriers requires new methods for retaining drugs of greater hydrophilicity inside of porous, nanostructured liquid and liquid crystalline phase materials, particularly at time points such as during coating processes and after coating dissolution/release. It is an object of this invention to provide such methods.

It is another object of this invention to provide a framework for a range of lipid-water systems and lipid-water-platinum drug systems that satisfy the four challenges listed above.

It is a further object of this invention to provide non-lamellar liquid crystalline materials that satisfy these four challenges and capitalize on the inherent advantages of non-lariellar liquid crystals and microparticles thereof. These advantages include bioadhesiveness, controllable porosity (e.g., for protection of internal components against degradative proteins), solubilization properties, and the potential for enhancement of cell uptake.

It is a further object of this invention to achieve solubilization of pharmaceutical actives which are otherwise challenging to solubilize in nanoporous, reversed liquid and liquid crystalline phase materials at pharmaceutically significant levels.

According to the invention, there is contemplated and utilized a complexation or ion-pairing of drugs, such as pharmaceutically-important platinum compounds, for solubilization and retention inside the interiors of nanoporous lipid-based matrices. The complexation or ion-pairing is with pharmaceutically-acceptable anions (or cations) that have high octanol-water partition coefficients, preferably greater than about 100 and more preferably greater than about 1,000, and/or which are a surfactant, particularly polar lipids that are a surfactant. By complexing or ion-pairing, the drug, or more precisely a cationic (anionic) moiety X that is a modification of the drug, solubility and partitioning properties can be dramatically altered, such that the four challenges listed above are met at once. Modification of the drug is typically by removal of a chloride (sodium) ion, and binding to a bilayer-associated anion (cation).

### DESCRIPTION OF THE DRAWINGS

FIG. 1 depicts one embodiment of the current invention, and schematically shows the cationic moiety 1 of a drug is ion-paired with the anionic moiety 2 of an anionic surfactant in the interior of a porous, reversed nanostructured material 3.

FIG. 2 depicts schematically, for the purpose of contrasting the current invention with the prior art, the situation that results when a cationic drug 4, together with its usual counterion 5, is incorporated into a nanostructured material 6 containing an anionic surfactant 7 (with its counterion 8).

FIG. 3 depicts schematically a hypothetical method, or “thought experiment”, which illustrates a fundamental difference between the current invention and a simple mixing of surfactant and drug.

### DETAILED DESCRIPTION OF THE PREFERRED EMBODIMENTS OF THE INVENTION

In the discussion of the present invention the definitions below will be used.

Definitions

Nanostructured: The terms “nanostructure” or “nanostructured” as used herein in the context of the structure of a material refer to materials the building blocks of which have a size that is on the order of nanometers (10⁻⁹ meter) or tens of nanometers (10×10⁻⁹meter). Generally speaking, any material that contains domains or particles 1 to 100 nm (nanometers) across, or layers or filaments of that thickness, can be considered a nanostructured material. (See also Dagani, R., “Nanostructured Materials Promise to Advance Range of Technologies.” Nov. 23, 1992 C&E News 18 (1992).) The term is meant to exclude so-called “ceramic glasses” which are crystalline materials in which the crystallite size is so small that one may not observe peaks in wide-angle x-ray diffraction and which some physicists may refer to as nanostructured materials. The nanostructured liquid and liquid crystalline phases that are defined herein are characterized by nanoscale domains which are clearly distiniguished from neighboring domains by large differences in local chemical composition, and do not include materials in which neighboring domains have essentially the same local chemical composition and differ only in lattice orientation. Thus, by the term “domain” as used herein, it is meant a spatial region which is characterized by a particular chemical makeup which is clearly distinguishable from that of neighboring domains. Often such a domain is hydrophilic (hydrophobic) which contrasts with the hydrophobicity (hydrophilicity) of neighboring domains. In the context of this invention, the characteristic size of these domains is in the nanometer range. The term “microdomain” is often used to indicate domains whose size range is micron or nanometer scale.

Very effective systems for satisfying such solubilization requirements are provided by lipid-water systems, in which microdomains are present which are very high in water content, and simultaneously hydrophobic domains are in very close contact with the aqueous domains. The presence of aqueous domains circumvents precipitation tendencies encountered in systems where water structure is interrupted by the presence of high loadings of co-solvents or co-solutes, as, for example, in concentrated aqueous polymer solutions. At the same time the proximity of hydrophobic domains provides for effective solubilization of amphiphilic compounds (and hydrophobic as well).

Nanostructured liquid and liquid crystalline phases are synthetic or semisynthetic materials which adopt these solubilization characteristics, and provide pure, well-characterized, easily produced, and typically inexpensive matrices that also have the following desirable properties:

a) versatility in chemical systems forming nanostructured liquid phases and nanostructured liquid crystalline phases, ranging from biological lipids that are ideal for biomolecules, to hardy fluorosurfactants, to glycolipids that bind bacteria, to surfactants with ionic or reactive groups, etc. This provides for applicability over a wide range of conditions and uses;

b) the unsurpassed ability of nanostructured liquid phases and nanostructured liquid crystalline phases to:

i) solubilize a wide range of active compounds including many traditionally difficult-to-solubilize compounds, circumventing the need for toxic and increasingly regulated organic solvents;

ii) achieve high concentrations of actives with uncompromised stability, and

iii) provide the biochemical environment that preserves their structure and function;

c) true thermodynamic stability, which greatly reduces instabilities common with other vehicles, such as precipitation of active agents, breaking of emulsions; and

d) the presence of a porespace with pre-selectable pore size in the nanometer range, facilitating further control of the release kinetics even after triggered release of the coating, particularly in the release of proteins and other biomacromolecules.

The desired properties of the nanostructured materials of focus herein derive from several related concepts regarding materials that can be described with respect to surfactants by use of the terms “polar,” “apolar,” “amphiphile,” “surfactant” and the “polar-apolar interface, and analogously with respect to block copolymer systems, as described below.

Polar: polar compounds (such as water) and polar moieties (such as the charged head groups on ionic surfactants or on lipids) are water-loving or hydrophilic. “Polar” and “hydrophilic” in the context of the present invention are essentially synonymous. In terms of polar groups in hydrophilic and amphiphilic molecules (including but not limited to polar solvents and surfactants), a number of polar groups are tabulated below.

Apolar: An apolar compound is a compound that has no dominant polar group. Apolar (or hydrophobic, or alternatively, “lipophilic”) compounds include not only the paraffinic/hydrocarbon/alkane chains of surfactants, but also modifications of them, such as perfluorinated alkanes, as well as other hydrophobic groups such as the fused-ring structure in cholic acid as found in bile salt surfactants, or phenyl groups as they form a portion of the apolar group in Triton-type surfactants, and oligomer and polymer chains that run the gamut from polyethylene (which represents a long alkane chain) to hydrophobic polymers such as hydrophobic polypeptide chains in novel peptide-based surfactants that have been investigated. A listing of some apolar groups and compounds is given below, in the discussion of useful components of the nanostructured phase interior. An apolar compound will be lacking in polar groups, a tabulation of which is included herein, and will generally have an octanol-water partition coefficient greater than about 100, and usually greater than about 1,000.

Amphiphile: an amphiphile can be defined as a compound that contains both a hydrophilic and a lipophilic group. See D. H. Everett. Pure and Applied Chemistry, vol. 31. no. 6, p. 611,1972. It is important to note that not every amphiphile is a surfactant. For example, butanol is an amphiphile, since the butyl group is lipophilic and the hydroxyl group hydrophilic, but it is not a surfactant since it does not satisfy the definition, given below. There exist a great many amphiphilic molecules possessing functional groups which are highly polar and hydrated to a measurable degree, yet which fail to display surfactant behavior. See R. Laughlin, Advances in liquid crystals, vol. 3. p. 41, 1978.

Surfactant: A surfactant is an amphiphile that possesses two additional properties. First, it significantly modifies the interfacial physics of the aqueous phase (at not only the air-water but also the oil-water and solid-water interfaces) at unusually low concentrations compared to nonsurfactants. Second, surfactant molecules associate reversibly with each other (and with numerous other molecules) to a highly exaggerated degree to form thermodynamically stable, macroscopically one-phase, solutions of aggregates or micelles. Micelles are typically composed of many surfactant molecules (10's to 1000's) and possess colloidal dimensions. See R. Laughlin, Advances in liquid crystals, vol. 3, p. 41, 1978. Lipids and polar lipids in particular, often are considered as surfactants for the purposes of discussion herein, although the term “lipid” is normally used to indicate that they belong to a subclass of surfactants which have slightly different characteristics than compounds which are normally called surfactants in everyday discussion. Two characteristics which frequently, though not always, are possessed by lipids are first, they are often of biological origin, and second, they tend to be more soluble in oils and fats than in water. Indeed, many compounds referred to as lipids have extremely low solubilities in water, and thus the presence of a hydrophobic solvent may be necessary in order for the interfacial tension-reducing properties and reversible self-association to be most clearly evidenced for lipids which are indeed surfactants. Thus, for example, such a compound will strongly reduce the interfacial tension between oil and water at low concentrations, even though extremely low solubility in water might make observation of surface tension reduction in the aqueous system difficult. Similarly, the addition of a hydrophobic solvent to a lipid-water system might make the determination of self-association into nanostructured liquid phases and nanostructured liquid crystalline phases a much simpler matter, whereas difficulties associated with high temperatures might make this difficult in the lipid-water system.

Indeed, it has been in the study of nanostructured liquid crystalline structures that the commonality between what had previously been considered intrinsically different, “lipids” and “surfactants”, came to the forefront, and the two schools of study (lipids, coming from the biological side, and surfactants, coming from the more industrial side) came together as the same nanostructures were observed in lipids as for all surfactants. In addition, it also came to the forefront that certain synthetic surfactants, such as dihexadecyldimethylammonium bromide, which were entirely of synthetic, non-biological origin, showed “lipid-like” behavior in that hydrophobic solvents were needed for convenient demonstration of their surfactancy. On the other end, certain lipids such as lysolipids, which are clearly of biological origin, display phase behavior more or less typical of water-soluble surfactants. Eventually, it became clear that for purposes of discussing and comparing self-association and interfacial tension-reducing properties, a more meaningful distinction was between single-tailed and double-tailed compounds, where single-tailed generally implies water-soluble and double-tailed generally oil soluble.

Thus, in the present context, any amphiphile which at very low concentrations lowers interfacial tensions between water and hydrophobe, whether the hydrophobe be air or oil, and which exhibits reversible self-association into nanostructured micellar, inverted micellar, or bicontinuous morphologies in water or oil or both, is a surfactant. The class of lipids simply includes a subclass consisting of surfactants which are of biological origin.

Polar-apolar interface: In a surfactant molecule, one can find a dividing point (or in some cases two points, if there are polar groups at each end, or even more than two, as in Lipid A, which has seven acyl chains and thus seven dividing points per molecule), in the molecule that divides the polar part of the molecule from the apolar part. In any nanostructured liquid phase or nanostructured liquid crystalline phase, the surfactant forms monolayer or bilayer films. In such a film, the locus of the dividing points of the molecules describes a surface that divides polar domains from apolar domains. This is called the “polar-apolar interface” or “polar-apolar dividing surface.” For example, in the case of a spherical micelle, this surface would be approximated by a sphere lying inside the outer surface of the micelle, with the polar groups of the surfactant molecules outside the surface and apolar chains inside it. Care should be taken not to confuse this microscopic interface with macroscopic interfaces separating two bulk phases that are seen by the naked eye.

Counterion: In the context of this invention, a counterion will be defined as a charged moiety that is part of a pharmaceutically-acceptable or pharmaceutically active salt or ion pair, such that another portion of the salt or ion pair is an organic moiety which contains the greater part of the organic portion of the overall compound. Thus, while the counterion may in fact be organic itself, such as a tartrate or citrate ion, the number of carbon atoms contained in the counterion will be significantly less than the number of carbon atoms in another portion of the compound with the opposite charge. Indeed, the number of carbon atoms in the counterion will nearly always be less than or equal to about six, and usually less than or equal to about 4. Conversely, essentially all surfactants, for example, have at least 8 carbon atoms. Indeed, in a pharmaceutical context, the most common counterions have no carbon atoms at all; the most common anionic counterions are chloride and bromide, with the next most common being tartrate, citrate, picrate, mesylate, maleate, and sulfate; the most common cationic counterions are sodium (Na⁺), potassium (K⁺), calcium (Ca²⁺), magnesium (Mg²⁺), ammonium, and protonated forms of low-carbon-number bases such as ethanolamine, diethanolamine, tromethamine, etc.; less common inorganic cationic counterions include ferrous, ferric, bismuth, zinc, and aluminum.

Cationic surfactant, anionic surfactant: In this disclosure, and as is well known in the art, a cationic surfactant is one in which the counterion is anionic, i.e., the greater part of the organic portion of the molecule is in the cationic moiety, and vice versa for an anionic surfactant.

Matrix: In the present context, a “matrix” is meant to be a material that serves as the host material for an active compound or compounds.

Moiety: A moiety in the present context is a chemical group that may, or (significantly) may not, exist as a stable, charge-neutral compound. Thus, for example, the stearate ion is a moiety that is a portion of the surfactant sodium stearate.

Bilayer-associated, membrane-associated: A compound or moiety is bilayer-associated if it partitions preferentially into a bilayer over an aqueous compartment. Thus, if a bilayer-rich material such as a lamellar phase or reversed cubic phase material exists in equilibrium with excess water and is placed in contact with excess water, and the compound or moiety allowed to equilibrate between the two phases, then the overwhelming majority of the compound or moiety will be located in the bilayer-rich phase. The concentration of the compound or moiety in the bilayer-rich phase will be at least about 100 times, and preferably at least about 1,000 times, larger than in the water phase.

Pharmaceutically-acceptable: In the context of this invention, “pharmaceutically-acceptable” generally designates compounds or compositions in which each excipient is approved by the Food and Drug Administration or a similar body in another country, for use in a pharmaceutical formulation intended for internal use. This also includes compounds that are major components of approved excipients, which are known to be of low toxicity taken internally. A listing of approved excipients, each with the various routes of administration for which they are approved, was published by the Division of Drug Information Resources of the FDA in January, 1996 and entitled “Inactive Ingredient Guide”. The existence of a Drug Master File at the FDA is additional evidence that a given excipient is acceptable for pharmaceutical use. In the present context, this listing includes, as approved for internal use (oral, injectable, intraperitoneal, etc.), such excipients as: benzyl benzoate, peppermint oil, orange oil, spearmint oil, ginger fluid extract (also known as essential oil of ginger), thymol, vanillin, anethole, cinnamon oil, cinnamaldehyde, clove oil, coriander oil, benzaldehyde, poloxamer 331 (Pluronic 101), polyoxyl 40 hydrogenated castor oil—indeed, a wide range of surfactants with polyethyleneglycol head groups—calcium chloride and docusate sodium. Absent from the list are a number of apolar or very weakly polar liquids that are more associated with applications as fuels or organic solvents: liquid hydrophobes including toluene, benzene, xylene, octane, decane, dodecane, and the like. In contrast, the hydrophobes and polar hydrophobes that are approved as excipients tend to be natural extracts which have a history of use in foods, nutriceuticals, or pharmaceutics—or early precursors to these disciplines. Examples of compounds that are major components of approved excipients and known to be of low toxicity include: linalool, which is a major component of coriander oil and is the subject of extensive toxicity studies demonstrating its low toxicity; vanillin, which is a major component of the approved excipient ‘flavor vanilla’ and is one of the major taste components of vanilla-flavored foods and pharmaceutical formulations; and d-limonene, which is a major component of the approved excipient ‘essence lemon’ approved for use in oral formulations and has extensive everyday applications in which its low toxicity is important. By “component” we mean a molecule that is present as a distinct and individual molecule in a mixture, not as a chemical group in a larger molecule; for example, methanol (methyl alcohol) would not be considered to be a component of methyl stearate. For the purposes of this invention, a compound will be considered to be a pharmaceutically-acceptable excipient if it can be created by a simple ion-exchange between two compounds that are on the FDA listing; thus, for example, calcium docusate is to be considered a pharmaceutically-acceptable excipient since it is a natural result of combining sodium docusate and calcium chloride (in the presence of water, for example). This does not extend, however, to compounds obtained by chemical reaction between two pharmaceutically-acceptable materials, since this may produce a material which is not pharmaceutically-acceptable.

Bicontinuous: In a bicontinuous structure, the geometry is described by two distinct, multiply-connected, intertwined subspaces each of which is continuous in all three dimensions. Thus, it is possible to traverse the entire span of this space in any direction even if the path is restricted to one or the other of the two subspaces. In a bicontinuous structure, each of the subspaces is rich in one type of material or moiety, and the two subspaces are occupied by two such materials or moieties each of which extends throughout the space in all three dimensions. Sponge, sandstone, apple, and many sinters are examples of relatively permanent though chaotic bicontinuous structures in the material realm. In these particular examples, one of the subspaces is occupied by a solid that is more or less deformable and the other subspace, though it may be referred to as void, is occupied by a fluid. Certain lyotropic liquid crystalline states are also examples, one subspace being occupied by amphiphile molecules oriented and aggregated into sheet-like arrays that are ordered geometrically, the other subspace being occupied by solvent molecules. Related liquid crystalline states that contain two incompatible kinds of solvent molecules, e.g. hydrocarbon and water, present a further possibility in which one subspace is rich in the first solvent, the other in the second, and the surface between lies within a multiply connected stratum rich in oriented surfactant molecules. Certain equilibrium microemulsion phases that contain comparable amounts of hydrocarbon and water as well as amphiphilic surfactant may be chaotic bicontinuous structures, maintained in a permanent state of fluctuating disorder by thermal motions, for they give no evidence of geometric order but there is compelling evidence for multiple continuity. Bicontinuous morphologies occur also in certain phase-segregated block copolymers. See Anderson. D. M., Davis. H. T., Nitsche. J. C. C. and Scriven. L. E. (1900) Advances in Chemical Physics, 77:337.

Dissolution: By the term “dissolution” is meant that a compound under consideration is dissolving, or is “undergoing dissolution”.

Solubilize: This term is meant to be essentially synonymous with the term “dissolve” or “dissolution”, though with a different connotation. A compound under consideration is solubilized in a liquid or liquid crystalline material if and only if the molecules of the compound are able to diffuse within the liquid or liquid crystalline material as individual molecules, and that such material with the compound in it make up a single thermodynamic phase. It should be borne in mind that slightly different connotations are associated with the terms “dissolve” and “solubilize”. Typically the term “dissolve” is used to describe the simple act of putting a crystalline compound in a liquid or liquid crystalline material and allowing or encouraging that compound to break up and dissolve in the material, whereas the terms “solubilize” and “solubilization” generally refer to a concerted effort to find an appropriate liquid or liquid crystalline material that is capable of dissolving such compound.

Association complex: For the purposes of this disclosure, two (or more) moieties are said to form an association complex if and only if they are bound together by the action of ionic (electrostatic) bonds and coordinate bonds but not traditional covalent bonds; thus, while hydrophobic interactions, hydrogen bonds, and other such relatively weak interactions may play a role in determining the overall strength and stability of the complex, the association must involve at least one ionic bond or one coordinate bond; and the binding must be limited to such bonds, so that the presence of a traditional (“non-coordinate”) covalent bond rules out the possibility of an association complex (as is well recognized in the art). Phrased otherwise, an association complex is formed by the association between a Lewis acid and a Lewis base, or in some cases this simplifies to the association between a simple acid and a simple base. The formation of a traditional covalent bond, in which a single orbital contains two electrons, one from each of the two atoms participating in the bond, is to be distinguished from a coordinate bond where both electrons in the bond are donated by only one of the atoms in the bond (typically a transition metal atom), the latter making for a more labile bond. From a pharmaceutical perspective, the more labile ionic and coordinate bonds represent much less of a departure from the original chemistry of the drug, such that pharmaceutical activity and toxicity are less profoundly modified and regulatory barriers for approval of the drug modification are significantly lower. As an example, in the case of a modification of the cisplatin molecule by coordinate bonding of an anionic organic moiety in place of a chloride, it is well established that relatively early in the pharmaceutical performance of cisplatin, the chloride is displaced by a water molecule anyway; thus, provided the organic moiety is similarly displaced by water in the body, in a simple aquation step, the active species will be the same in either case; the crucial point is that in either case, there is no need for enzymatic activity, in particular, in order to displace either the chloride or the organic anion. This is in contrast to the case of a prodrug, in which a classical covalent bond must be cleaved, typically by enzymatic action, in order to create the active species in the body. Such requirements in the case of prodrugs not only give rise to larger variations (both intersubject and intrasubject) in pharmacokinetics and/or pharmacodynamics, but also they create more complicated and expensive regulatory issues. The present invention, surprisingly, provides a means to prevent or greatly reduce leakage of drug from useful bilayer-based nanoporous matrices by the application of surfactants and other bilayer-associated (and even bilayer-forming) components in ways that avoid covalent modification of the drug and, at least in some cases, without the creation of new chemical entities (NCE's). For a reference discussing ionic association complexes, see T. Naito, Y. Tsuiki and H. Yamada, Analytical Sciences (2001), vol. 17, page 291.

Chemical criteria: A number of criteria have been tabulated and discussed in detail by Robert Laughlin for determining whether a given polar group is functional as a surfactant head group, where the definition of surfactant includes the formation in water of nanostructured phases even at rather low concentrations. R. Laughlin, Advances in Liquid Crystals, pp. 3-41, 1978.

The following listing given by Laughlin gives some polar groups which are not operative as surfactant head groups are: aldehyde, ketone, carboxylic ester, carboxylic acid, isocyanate, amide, acyl cyanoguanidine, acvl guanyl urea, acyl biuret, N.N-dimethylamide, nitrosoalkane, nitroalkane, nitrate ester, nitrite ester, nitrone, nitrosamine, pyridine N-oxide, nitrile, isonitrile, amine borane, amine haloborane, sulfone, phosphine sulfide, arsine sulfide, sulfonamide, sulfonamide methylimine, alcohol (monofunctional), ester (monofunctional), secondary amine, tertiary amine, mercaptan, thioether, primary phosphine, secondary phosphine, and tertiary phosphine. Thus, for example, an alkane chain linked to one of these polar groups would not be expected to form nanostructured liquid or liquid crystalline phases

Some polar groups which are operative as surfactant head groups, and thus, for example, an alkane chain linked to one of these polar groups would be expected to form nanostructured liquid and liquid crystalline phases, are:

a. Anionics: carboxylate (soap), sulfate, sulfamate, sulfonate, thiosulfate, sulfinate, phosphate, phosphonate, phosphinate, nitroamide, tris(alkylsulfonyl)methide, xanthate;

b. Cationics: ammonium, pyridinium, phosphonium, sulfonium, sulfoxonium;

c. Zwiterionics: ammonio acetate, phosphoniopropane sulfonate, pyridinioethyl sulfate; and

d. Semipolars: amine oxide, phosphonyl, phosphine oxide, arsine oxide, sulfoxide, sulfoximine, sulfone diimine, ammonio amidate.

Laughlin also demonstrates that as a general rule, if the enthalpy of formation of a 1:1 association complex of a given polar group with phenol (a hydrogen bonding donor) is less than 5 kcal, then the polar group will not be operative as a surfactant head group.

It is very important to point out that for nearly all the operative anionic polar groups, the protonated form (if it exists) is not operative as a head group. Thus, for example, fatty acids are not surfactants, whereas their sodium salts are (if the chain length is in the correct range). Phrased otherwise, in the terminology of some surfactant scientists, a protonated acidic group on an amphiphilic molecule does not constitute a water-soluble “head”. This is the reason why, in the discussions of counterions contained herewithin, the proton is not included as a potential counterion. The properties of the metal salts of organic anions, particularly the salts with monovalent metal ions, are vastly different from those of the corresponding protonated organic anion, in terms of solubility, partitioning, bilayer interactions, association behavior, and a wide range of other thermodynamic properties.

In addition to the polar head group, a surfactant requires an apolar group. Again, there are guidelines for an effective apolar group. For alkane chains, which are of course the most common, if n is the number of carbons, then n must be at least 6 for surfactant association behavior to occur, although at least 8 or 10 is the usual case. Interestingly octylamine, with n=8 and the amine head group which is just polar enough to be effective as a head group, exhibits a lamellar phase with water at ambient temperature, as well as a nanostructured L2 phase. Warnhelm. T., Bergenstahl. B., Henriksson. U., Malmvik. A.-C. and Nilsson. P. (1987) J. of Colloid and Interface Sci. 118:233. Branched hydrocarbons yield basically the same requirement on the low n end: for example, sodium 2-ethylhexylsulfate exhibits a full range of liquid crystalline phases. Winsor, P. A. (1968) Chem. Rev. 68:1. However, the two cases of linear and branched hydrocarbons are vastly different on the high n side. With linear, saturated alkane chains, the tendency to crystallize is such that for n greater than about 18, the Krafft temperature becomes high and the temperature range of nanostructured liquid and liquid crystalline phases increases to high temperatures, near or exceeding 100° C. In the context of the present invention, for most applications this renders these surfactants considerably less useful than those with n between 8 and 18. With the introduction of unsaturation or branching in the chains, the range of n can increase dramatically. The case of unsaturation can be illustrated with the case of lipids derived from fish oils, where chains with 22 carbons can have extremely low melting points due to the presence of as many as 6 double bonds, as in docosahexadienoic acid and its derivatives, which include monoglycerides, soaps, etc. Furthermore, polybutadiene of very high MW is an elastomeric polymer at ambient temperature, and block copolymers with polybutadiene blocks are well known to yield nanostructured liquid crystals. Similarly, with the introduction of branching one can produce hydrocarbon polymers such as polypropyleneoxide (PPO) which serves as the hydrophobic block in a number of amphiphilic block copolymer surfactants of great importance, such as the Pluronic series of surfactants. Substitution of fluorine for hydrogen, in particular the use of perfluorinated chains, in surfactants generally lowers the requirement on the minimal value of n, as exemplified by lithium perfluourooctanoate (n=8), which displays a full range of liquid crystalline phases, including an intermediate phase which is fairly rare in surfactant systems. As discussed elsewhere, other hydrophobic groups, such as the fused-ring structure in the cholate soaps (bile salts), also serve as effective apolar groups, although such cases must generally be treated on a case by case basis in terms of determining whether a particular hydrophobic group will yield surfactant behavior.

For single-component block copolymers, relatively simple mean-field statistical theories are sufficient to predict when nanostructure liquid phase and liquid crystalline phase materials will occur and these are quite general over a wide range of block copolymers. If chi is the Flory-Huggins interaction parameter between polymer blocks A and B, and N is the total index of polymerization defined as the number of statistical units or monomer units in the polymer chain, consistently with the definition of the interaction parameter of the block copolymer, then nanostructure liquid and liquid crystalline phases are expected when the product of chi and N is greater than 10.5. Leibler, L. (1980) Macromolecules 13:1602. For values comparable to but larger than this critical value of 10.5, ordered nanostructured (liquid crystalline) phases can occur, including ever, bicontinuous cubic phases. See Hajduk,. D. A., Harper, P. E., Gruner, S. M., Honeker, C. C., Kim, G., Thomas, E. L. and Fetters, L. J. (1994) Macromolecules 27:4063.

L3 phase: L2-phase regions in phase diagrams sometimes exhibit “tongues” sticking out of them. These are long, thin protrusions unlike the normal appearance of a simple L2 phase region. This sometimes appears also with some L1 regions, as described below. When one examines these closely, especially with X-ray and neutron scattering, they differ in a fundamental way from L2 phases. In an L2 phase, the surfactant film is generally in the form of a monolayer with oil (apolar solvent) on one side and water (polar solvent) on the other. By contrast, in this “L3 phase” as these phases are called, the surfactant is in the form of a bilayer with water (polar solvent) on both sides. The L3 phase is generally considered to be bicontinuous and, in fact, it shares another property with cubic phases: there are two distinct aqueous networks interwoven but separated by the bilayer. So, the L3 phase is really very similar to the cubic phase but lacking the long-range order of the cubic phase. L3 phases stemming from L2 phases and those stemming from L1 phases are given different names. “L3 phase” is used for those associated to L2 phases, and “L3*phase” for those associated to L1 phases.

Determination of the L3 phase in distinction to the other liquid phases discussed herein can be a sophisticated problem, requiring the combination of several analyses. The most important of these techniques are now discussed. In spite of its optical isotropy when acquiescent and the fact that it is a liquid, the L3 phase can have the interesting property that it can exhibit flow birefringence. Often this is associated with fairly high viscosity, e.g., viscosity that can be considerably higher than that observed in the L1 and L2 phases, and comparable to or higher than that in the lamellar phase. These properties are of course a result of the continuous bilayer film, which places large constraints on the topology and the geometry of the nanostructure. Thus, shear can result in the cooperative deformation (and resulting alignment) of large portions of the bilayer film, in contrast with, for example, a micellar L1 phase where independent micellar units can simply displace with shear. In any case, a monolayer is generally much more deformable under shear than a bilayer. Support for this interpretation comes from the fact that the viscosity of L3 phases is typically a linear function of the volume fraction of surfactant. Snabre. P. and Porte. G. (1990) Europhys. Len. 13:641.

Sophisticated light, neutron, and x-ray scattering methodologies have been developed for determination of nanostructured L3 phases. Safinya, C. R., Roux, D., Smith,. G. S., Sinha, S. K., Dimon, P., Clark, N. A. and Bellocq, A. M. (1986) Phys. Rev. Lett. 57:2718; Roux, D. and Safinya, C. R. (1988) J. Phys. France 49:307; Nallet, F., Roux, D. and Prost, J. (1989) J. Phys. France 50:3147. The analysis of Roux, et al. in Roux, D., Cates, M. E., Olsson, U., Ball, R. C., Nallet, F. and Bellocq, A. M., Europhys. Lett. With these methodologies, it is possible to determine that the nanostructure has two aqueous networks, separated by the surfactant bilayer, which gives rise to a certain symmetry due to the equivalence of the two networks.

Fortunately, determination of the nanostructured nature of an L3 phase based on phase behavior can be more secure than in the case of typical L1, L2, or even microemulsion phases. This is first of all because the L3 phase is often obtained by addition of a small amount (a few percent) of oil or other compound to a lamellar or bicontinuous cubic phase, or small increase of temperature to these same phases. Since these liquid crystalline phases are easy to demonstrate to be nanostructured (Bragg peaks in X-ray, in particular), one can be confident that the liquid phase is also nanostructured when it is so close in composition to a liquid crystalline phase. After all, it would be extremely unlikely that the addition of a few percent of oil to a nanostructured liquid crystalline phase would convert the liquid crystal to a structureless liquid. Indeed, pulsed-gradient NMR self-diffusion measurements in the Aerosol OT—brine system show that the self-diffusion behavior in the L3 phase extrapolates very clearly to those in the nearby reversed bicontinuous cubic phase. This same L3 phase has been the subject of a combined SANS, self-diffusion, and freeze-fracture-electron microscopy study. Strey, R., Jahn,. W., Skouri, M., Porte, G., Marisman,. J. and Olsson,. U. in “Structure and Dynamics of Supramolecular Aggregates—S. H. Chen, J. S. Huang and P. Tartaglia, Eds., Kluwer Academic Publishers, The Netherlands. Indeed, in SANS and SAXS scattering analysis of L3 phases, a broad interference peak is often observed at wave vectors that correspond to d-spacings that are the same order of magnitude as those in bicontinuous cubic phases that are nearby in the phase diagram, and the author has developed a model for L3 phase nanostructure which is an extrapolation of known structures for bicontinuous cubic phases. Anderson, D. M., Wennerstrom, H. and Olsson, U. (1989) J. Phys. Chem. 93:4532.

The nanostructured liquid crystalline phases are characterized by domain structures composed of domains of at least a first type and a second type (and in some cases three or even more types of domains) having the following properties:

a) the chemical moieties in the first type domains are incompatible with those in the second type domains (and in general, each pair of different domain types are mutually incompatible) such that they do not mix under the given conditions but rather remain as separate domains (for example, the first type domains could be composed substantially of polar moieties such as water and lipid head groups, while the second type domains could be composed substantially of apolar moieties such as hydrocarbon chains: or, first type domains could be polystyrene-rich, while second type domains are polyisoprene-rich, and third type domains are polyvinylpyrrolidone-rich);

b) the atomic ordering within each domain is liquid-like rather than solid-like, lacking lattice-ordering of the atoms (this would be evidenced by an absence of sharp Bragg peak-reflections in wide-angle x-ray diffraction);

c) the smallest dimension (e.g., thickness in the case of layers, diameter in the case of cylinders or spheres) of substantially all domains is in the range of nanometers (viz., from about 1 to about 100 nm); and

d) the organization of the domains conforms to a lattice, which may be one-, two-, or three-dimensional and which has a lattice parameter (or unit cell size) in the nanometer range (viz., from about 5 to about 200 nm), the organization of domains thus conforms to one of the 230 space groups tabulated in the International Tables of Crystallography and would be evidenced in a well-designed small-angle x-ray scattering (SAXS) measurement by the presence of sharp Bragg reflections with d-spacings of the lowest order reflections being in the range of 3-200 nm.

Reversed hexagonal phase: In surfactant-water systems, the identification of the reversed hexagonal phase is as follows:

1. Small-angle x-ray shows peaks indexing as 1:√3:2:√7:3 . . . ; in general, √(h²+hk+k²), where h and k are integers—the Miller indices of the two-dimensional symmetry group.

2. To the unaided eye, the phase is generally transparent when fully equilibrated, and thus often considerably clearer than any nearby lamellar phase.

3. In the polarizing optical microscope, the phase is birefringent, and the well-known textures of hexagonal phases (which apply to both normal and reversed types) have been well described by Rosevear, and by Winsor (e.g., Chem. Rev. 1968, p.1). The most distinctive of these is the “fan-like” texture. This texture appears to be made up of patches of birefringence, where within a given patch, fine striations fan out giving an appearance reminiscent of an oriental fan. Fan directions in adjacent patches are randomly oriented with respect to each other. A key difference distinguishing between lamellar and hexagonal patterns is that the striations in the hexagonal phase do not, upon close examination at high magnification, prove to be composed of finer striations running perpendicular to the direction of the larger striation, as they do in the lamellar phase.

For reversed hexagonal phases in surfactant-water systems:

1. Viscosity is moderate to very high, more viscous than the lamellar phase and often as viscous as the reversed cubic phases (which have viscosities in the millions of centipoise).

2. The self-diffusion coefficient of water is slow compared to that in the lamellar phase; that of the surfactant is comparable to that in the lamellar phase.

3. The ²H NMR bandshape using deuterated surfactant shows a splitting, which is one-half the splitting observed for the lamellar phase.

4. In terms of phase behavior, the reversed hexagonal phase generally occurs at high surfactant concentrations in double-tailed surfactant/water systems, often extending to, or close to, 100% surfactant. Usually the reversed hexagonal phase region is adjacent to the lamellar phase region which occurs at lower surfactant concentration, although bicontinuous reversed cubic phases often occur in between. The reversed hexagonal phase does appear, somewhat surprisingly, in a number of binary systems with single-tailed surfactants, such as those of many monoglycerides (include glycerol monooleate), and a number of nonionic PEG-based surfactants with low HLB.

For hexagonal phases in single-component block copolymer systems, the terms “normal” and “reversed” do not generally apply (although in the case where one block is polar and the other apolar, these qualifiers could be applied in principle). The shear modulus in such a hexagonal phase is generally higher than a lamellar phase, and lower than a bicontinuous cubic phase, in the same system. In terms of phase behavior, the hexagonal phases generally occurs at volume fractions of the two blocks on the order of 35:65. Typically, two hexagonal phases will straddle the lamellar phase, with, in each case, the minority component being inside the cylinders (this description replacing the ‘normal/reversed’ nomenclature of surfactant systems).

Reversed cubic phase: This is defined to be either a reversed bicontinuous cubic phase, or a reversed discrete cubic phase, both of which are defined below.

Reversed bicontinuous cubic phase: The reversed bicontinuous cubic phase is characterized by:

1. Small-angle x-ray shows peaks indexing to a three-dimensional space group with a cubic aspect. The most commonly encountered space groups, along with their indexings, are: Ia3d (#230), with indexing √6:√8:√14:4: . . . ;Pn3m (#224), with indexing √2√:3:2:√6:√8: . . . ; and Im3m (#229), with indexing √2:√4:√6:√8:√10: . . .

2. To the unaided eye, the phase is generally transparent when fully equilibrated, and thus often considerably clearer than any nearby lamellar phase.

3. In the polarizing optical microscope, the phase is non-birefringent, and therefore there are essentially no optical textures.

For reversed bicontinuous cubic phases in surfactant-water systems:

1. Viscosity is high, much more viscous than the lamellar phase. Most reversed cubic phase have viscosities in the millions of centipoise.

2. No splitting is observed in the NMR bandshape, only a single peak corresponding to isotropic motion.

3. In terms of phase behavior, the reversed bicontinuous cubic phase is found between the lamellar phase and the reversed hexagonal phase, whereas the normal is found between the lamellar and normal hexagonal phases. One must therefore make reference to the discussion above for distinguishing normal hexagonal from reversed hexagonal. A good rule is that if the cubic phase lies to higher water concentrations than the lamellar phase, then it is normal, whereas if it lies to higher surfactant concentrations than the lamellar then it is reversed. The reversed cubic phase generally occurs at high surfactant concentrations in double-tailed surfactant/water systems, although this is often complicated by the fact that the reversed cubic phase may only be found in the presence of added hydrophobe (“oil”) or amphiphile. The reversed bicontinuous cubic phase does appear in a number of binary systems with single-tailed surfactants such as those of many monoglycerides (include glycerol monooleate) and a number of nonionic PEG-based surfactants with low HLB.

For bicontinuous cubic phases in single-component block copolymer systems, the terms “normal” and “reversed” do not generally apply (although in the case where one block is polar and the other apolar, these qualifiers could be applied in principle). The shear modulus in such a bicontinuous cubic phase is generally much higher than a lamellar phase, and significantly than a hexagonal phase, in the same system. In terms of phase behavior, the bicontinuous cubic phases generally occur at volume fractions of the two blocks on the order of 26:74. In some cases, two bicontinuous cubic phases will straddle the lamellar phase, with, in each case, the minority component being inside the cylinders (this description replacing the ‘normal/reversed’ nomenclature of surfactant systems), and hexagonal phases straddling the cubic-lamellar-cubic progression.

Self-diffuision coefficients of all components are comparable to those in the lamellar phase (except in some cases, where the diffusion of water can become very low if the water content is very low).

It should also be noted that in reversed bicontinuous cubic phases, though not in normal, the space group #212 has been observed. This phase is derived from that of space group #230.

Reversed discrete cubic phase: The reversed discrete cubic phase is characterized by:

1. Small-angle x-ray shows peaks indexing to a three-dimensional space group with a cubic aspect. The most commonly encountered space group in surfactant systems is Pm3n (#223), with indexing √2:√4:√5: . . . . In single-component block copolymers, the commonly observed space group is Im3m, corresponding to body-centered, sphere-packings, with indexing √2:√4:√6:√8: . . .

2. To the unaided eye, the phase is generally transparent when fully equilibrated, and thus often considerably clearer than any associated lamellar phase.

3. In the polarizing optical microscope, the phase is non-birefringent, and therefore there are essentially no optical textures.

For reversed discrete cubic phases in surfactant-water systems:

1. Viscosity is high, much more viscous than the lamellar phase and even more viscous than typical normal hexagonal phases. Most cubic phase have viscosities in the millions of centipoise, whether discrete or bicontinuous.

2. Also, in common with the bicontinuous cubic phases, there is no splitting in the NMR bandshape, only a single isotropic peak.

3. In terms of phase behavior, the reversed discrete cubic phase is found between the lamellar phase and the reversed hexagonal phase, whereas the normal is found between the lamellar and normal hexagonal phases. One must therefore make reference to the discussion above for distinguishing normal hexagonal from reversed hexagonal. A good rule is that if the cubic phase lies to higher water concentrations than the lamellar phase, then it is normal, whereas if it lies to higher surfactant concentrations than the lamellar then it is reversed. The reversed cubic phase generally occurs at high surfactant concentrations in double-tailed surfactant/water systems, although this is often complicated by the fact that the reversed cubic phase may only be found in the presence of added hydrophobe (“oil”) or amphiphile. The reversed discrete cubic phase does appear in a number of binary systems with single-tailed surfactants, such as those of many monoglycerides (include glycerol monooleate), and a number of nonionic PEG-based surfactants with low HLB.

4. The space group observed is usually Fd3m. #227.

5. The self-diffusion of the water is very low, while that of any hydrophobe present is high; that of the surfactant is generally fairly high, comparable to that in the lamellar phase. As stated above in the discussion of normal discrete cubic phases, the distinction between “normal” and “reversed” discrete cubic phases makes sense only in surfactant systems, and generally not in single-component block copolymer discrete cubic phases.

The Invention

The basis for this invention is the complexation or ion-pairing of drugs, such as pharmaceutically-important platinum compounds, for solubilization and retention inside the interiors of nanoporous lipid-based matrices. The complexation or ion-pairing is with pharmaceutically-acceptable anions (or cations) that have high octanol-water partition coefficients, preferably greater than about 100 and more preferably greater than about 1,000, and/or which satisfy the definition of a surfactant, particularly polar lipids that satisfy the definition of a surfactant (given below). By complexing or ion-pairing the drug, or more precisely a cationic (anionic) moiety X that is a modification of the drug, typically by removal of a chloride (sodium) ion, to a bilayer-associated anion (cation), the solubility and partitioning properties of the drug can be dramatically altered, such that the four challenges listed above are met at once. To begin with, the solubility of the drug in lipid-water systems can be dramatically improved, because due to the electrostatic attraction between X and the anion (cation), X is substantially bound to the anion (cation) and “goes along for the ride” in the solubilization of the anion (cation), and thus the complex, in the bilayer. For the same reason, the partitioning of the anion (cation) into lipophilic regions can also carry along the cation (anion) X, during encapsulation and during the transit in the body. Significantly, in the case of a platinum drug, the presence of an anion that is much bulkier than a chloride ion can serve to sterically inhibit attack by thiol compounds, particularly if the anion has a substantial hydrophobic portion.

It is important to point out that the above description refers to a complex or salt between the cationic (anionic) portion of the drug—in particular, absent the usual anionic (cationic) counterion—and a bilayer-associated anion, that is, the anionic (cationic) portion of a surfactant, in particular, absent the usual cationic (anionic) counterion. This approach avoids an important pitfall that is encountered when one simply mixes drug (with counterion present) and surfactant (i.e., with counterion present). The pitfall is that the presence of cationic and anionic counterions interrupts electrostatic interactions between the drug and surfactant, and in fact renders such interactions intermittent, effectively. Consider, for example, the case of an anionic drug, say with a carboxyl group, which is typical for an anionic drug. Assume that a cationic surfactant, say the chloride salt of a quaternary ammonium surfactant, is used in an attempt to bind the drug. Such a surfactant would not significantly increase the degree of dissociation of the carboxyl group, and with a typical pKa of around 4.5, at any given moment only a small fraction (on the order of 1%) of the drug would be charged at all, in the absence of buffer. In the presence of buffer, say significantly above pH 4.5, the drug would be nearly always charged, but the intended quaternary ammonium group of the surfactant would face strong competition from the buffer cations (typically Na⁺) for association with the charged carboxylate group. And the Debye length would be small compared to the unbuffered system (and especially compared to a system of the current invention, where the surfactant and drug counterions have been removed), thus screening electrostatic interactions generally. Thus, in the current invention, the direct interaction between drug cation and surfactant anion provides for a much stronger and more permanent binding of drug to matrix, than would a simple mixture of drug and surfactant (with their respective counterions intact). In particular, if a porous liquid crystalline particle containing a complex of the current invention were placed in pure water, drug could not leak out of the particle without violating charge neutrality of the particle, which is extremely unfavorable thermodynamically—indeed, such a charge imbalance would lead, literally, to an explosion, and simply does not occur in chemical systems suitable for pharmaceutical application.

The approach described above can be adapted to a wide range of drugs. In particular, the following two general types of compositions fall within the scope of the current invention:

1) A reversed cubic or reversed hexagonal or L3 phase material comprising a pharmaceutical active that is an association complex between two moieties, wherein one of these moieties consists essentially of one or more anionic compounds, and wherein for substantially every such anionic compound forms a pharmaceutically acceptable anionic surfactant with at least one cationic counterion which is different from the two aforementioned moieties; and

2) A reversed cubic or reversed hexagonal or L3 phase material comprising a pharmaceutical active that is an association complex between two moieties at least one of which itself is pharmaceutically active and is larger than one element in size (e.g., lithium and magnesium), wherein one of these moieties consists essentially of one or more cationic compounds, and wherein for substantially every such cationic compound forms a pharmaceutically acceptable cationic surfactant with at least one anionic counterion which is different from the two aforementioned moieties.

This can be further generalized within the scope and context of the current invention, by using, instead of surfactants, compounds that have high octanol-water partition coefficients, preferably greater than about 100 and more preferably greater than about 1,000. Such a compound, when bound through a coordinate bond or ionic bond to a drug moiety, will provide a substantial retention of the drug within the porous material by virtue of the hydrophobic interaction with the lipid or surfactant monolayer.

Similarly as in the formation of a coordination complex between the two moieties, the formation of an ionic bond, or salt, between the two moieties for retention in a nanoporous material also calls for removal of the typical counterions that are present when combining a standard pharmaceutical surfactant with a drug compound; for example, in combining benzalkonium chloride with sodium alendronate, the chloride and sodium counterions must be eliminated.

Very significantly, the binding of the drug moiety to a lipid in the vehicle via electrostatic interactions means that the lipid matrix can be porous, in sharp contrast with the case without this electrostatic binding where porosity would allow leakage of the drug out of the vehicle and would thus be precluded. Porous lipid phases such as reversed hexagonal phases and, in particular, reversed cubic phases, are well suited for enhancing direct, fusion-mediated cellular uptake. Furthermore, from a processing standpoint, the high viscosities of the “semi-solid” reversed cubic and hexagonal phase materials makes them well suited for many processes such as microencapsulation, etc.

In particular, in U.S. Pat. No. 6,482,517, the entire contents of which are hereby incorporated by way of reference, the current author has described microencapsulation systems incorporating lipid-based lyotropic liquid crystals, in which nanoporous hexagonal and, in particular, cubic phases are of central importance. The porous nature of these phases, and the interrelationship between this porosity and the lipid monolayer curvature properties which tend to promote fusion between these phases and bilayers (in particular, biomembranes), make them well suited for promoting cellular uptake and circumventing endosomal entrapment and other limitations that liposomes, for example, face. However, this same porosity can result in drug leakage, even in the case of coated particles, because during certain stages in production and in application the coating can be incomplete or dissolved. Therefore the present invention can be of considerable value in such systems, in relieving problems associated with drug that is not strongly bound to the matrix material.

It should be pointed out that from a regulatory perspective, the association complexes formed in this invention may have a different regulatory status than the starting materials, namely the drug and the surfactant or high-Kow compound. This further underscores the fact that removal of the counterions and direct complexation or ion-pairing between the (counterion-free) moieties constitutes an approach that is fundamentally different from simply mixing drug and surfactant.

Methods and Materials

In the general process, an appropriate anionic component is first selected based on such properties as partition coefficient (generally high is best, preferably greater than about 1,000), low toxicity, favorable regulatory status, melting point, and solubility/compatibility with the other components of the formulation. A number of methods can be used to bind this anion to the cationic platinum moiety. One particularly useful and straightforward method is to replace one or more chloride ions on the platinum compound with nitrate ions, by dissolving the drug and silver nitrate in water, alcohol, or other suitable solvent and precipitating silver chloride. The nitrate ions are then easily displaced by many of the anionic groups listed above, particularly those strong enough to serve as polar head groups. This displacement can be performed in a common solvent, such as alcohol, or in some cases in a lipid system that incorporates other components of the final lipid-based formulation. While the formulation should exhibit compatibility between the various lipids used, it is entirely possible to use one (anionic) lipid for the complexation with the drug, and a second lipid or lipid mixture for the majority component of the lipid matrix. For example, ethylhexylsulfosuccinate (docusate) can be used to bind the drug while phosphatidylcholine is the main component of the matrix.

A number of methods are known in organic chemistry for performing the elimination of counterions and forming salts, or ion pairs, between organic moieties. One method is to replace the cation with a proton, and the anion with an OH— group, and then combining the two to form water as a condensation product. A short-chain alcohol, such as ethanol, with dissolved acid (e.g., hydrochloric acid) can be used to replace the cation with a proton, with the precipitation of a simple salt such as sodium chloride. Similary, NaOH dissolved in ethanol can replace the anion with an OH— group. After removal of the precipitated salt, and in some cases with the subsequent removal of the solvent, the protonated and hydroxylated compounds can then be combined, often in aqueous solution. A variation of this method that sometimes works is to mix the two compounds with their respective counterions in a solvent that is a non-solvent for the salt formed by the two counterions—typically ethanol, which is a non-solvent for such simple salts as sodium chloride but often a solvent for both the starting compounds and the final ion-paired compound. Another method is to use an ion-exchange resin. For example, a cation-exchange resin can be charged with the cationic moiety of interest, after which the anionic moiety in either protonated or salt form is incubated with the exchange resin.

The incorporation of the drug-lipid complex in the liquid crystal follows the same procedures as used in the solubilization of any compound in a liquid crystal, which is described in U.S. Pat. No. 6,482,517. In short, this is performed by mixing the drug-lipid complex with the other components and allowing equilibration, with due attention paid to the phase behavior that the components together display, which in turn is determined by polarizing optical microscopy, viscosity features, and small-angle x-ray when necessary. The same patent describes the production and characterization of coated microparticles with one of these materials serving as the core of the microparticle, for application to drug delivery, including targeted delivery. U.S. Pat. No. 5,531,925 also describes microparticles, in this case uncoated, based on non-lamellar lyotropic liquid crystalline phases, for drug delivery, possibly including platinum compounds; in the case of uncoated particles such as these, the complexation of a platinum drug would be of high importance because the absence of a coating calls for another method to retain the drug inside the particles during production, storage, and during transit in the body.

Another related method applies to platinum drugs that are not amenable to the above method, typically because they are insoluble in water and alcohol. It is in fact common for platinum drugs to be of very low solubility in virtually all common solvents except for DMSO and members of the formamide and acetamide series. Indeed, this fact is one important motivating factor for the present invention. The drug is first dissolved in one of these solvents, preferably dimethylacetamide because this solvent is of low toxicity and is used in currently marketed drug formulations; furthermore, it is a solvent for silver nitrate. For the purposes of this discussion, we will assume that dimethylacetamide is used. Silver nitrate (preferably pre-dissolved in the same solvent) can then be added if desired, to convert the chloride to nitrate as above. The anionic compound is then added to the solution, promoting the formation of the desired complex, and at this point the other components of the lipid-based matrix can be added. The addition of these components, or even of just the anionic compound, can result in a multiphase system, for example a liquid crystalline phase in equilibrium with an excess solvent-rich liquid. However, for the purposes of creating a drug-anion complex, this is of secondary importance, since the complex is designed to partition into the lipid-rich phase, and provided sufficient mixing and/or equilibration of the phase(s) is applied, the complex will form and partition correctly. Nevertheless, to ensure that the drug ends up primarily in the lipid-rich phase, it can be useful to add water, glycerol, or other polar solvent to the (more amphiphilic) dimethylacetamide. One useful approach is to pre-mix the major components of the lipid-water phase, to accomplish the hydration of the lipid, before combining with the dimethylacetamide mixture. Ultimately, it can be important to remove the dimethylacetamide (or at least most of it), and this can be accomplished by essentially washing the liquid crystal (or other lipid-water matrix) containing the complex with water, glycerol, or other polar solvent, because the lipid-water matrix will in general be chosen so as to be insoluble in water (and/or other polar solvents). Alternatively, processes such as diafiltration, dialysis, and the like can be applied.

In the case of liposome-based vehicles, the production of liposomes, and the incorporation of lipids and related compounds is well known in the art. Such techniques can be applied to the incorporation of the anion-drug complexes described in this invention. For example, in the methodology described in the previous paragraph, after the removal of dimethylacetamide, the (hydrated) lipid mixture can be sonicated, or homogenized, in the presence of water, provided the composition is such that a lamellar liquid crystalline phase is present and capable of forming liposomes. However, such liposomal materials are not considered as falling within the current invention, because hydrophilic compounds are entrapped in liposomes simply by virtue of the geometry, due to the high resistance to transit across bilayers for such materials. Indeed, complexing or ion-pairing a hydrophilic drug, which otherwise cannot easily cross a bilayer, with a surfactant moiety could actually provide a mechanism for the drug to cross the bilayer and escape the liposome.

The phases that can be in equilibrium with water are preferred from the point of view of making coated particles of the present invention. A number of reversed cubic, reversed hexagonal, and L3 phases in fact have this property. Preferably, in using the process described herein to disperse a given phase as the matrix, it is desirable that the phase be insoluble in water, or whatever solvent the particles are dispersed in. Furthermore, when the interior phase has the additional property that it is in equilibrium with excess aqueous solution during formation of the particles, then concerns of phase transformation are minimized. Similarly when the interior phase is in equilibrium with excess aqueous solution under the conditions encountered when and after the particle coating is released, then the concerns of phase changes are likewise minimized, and in some applications this may be advantageous.

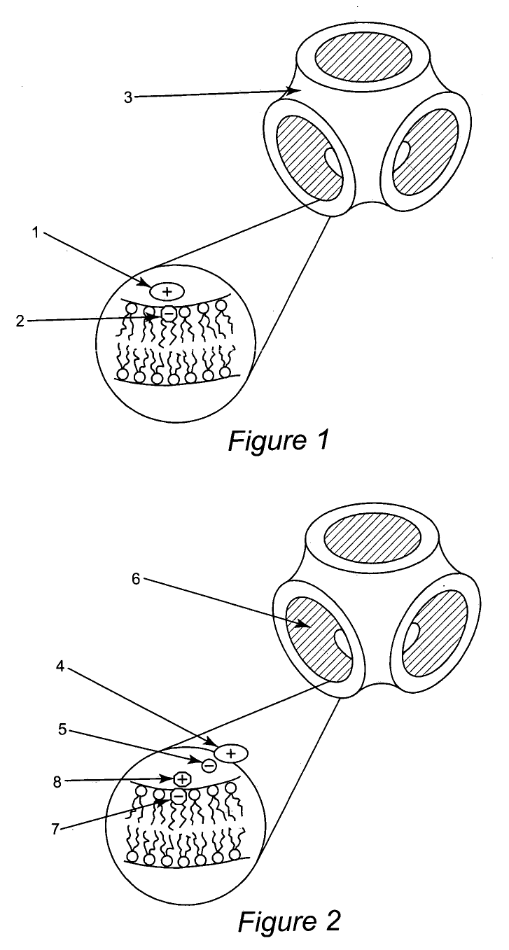

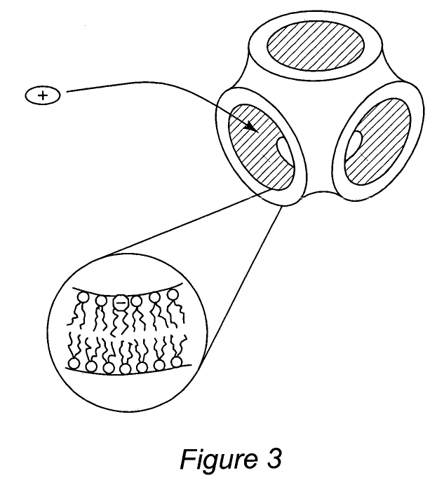

With reference to the drawings, FIG. 1 depicts one embodiment of the current invention. The cationic moiety 1 of a drug is ion-paired with the anionic moiety 2 of an anionic surfactant in the interior of a porous, reversed nanostructured material 3. FIG. 2 depicts, for the purpose of contrasting the current invention with the prior art, the situation that results when a cationic drug 4, together with its usual counterion 5, is incorporated into a nanostructured material 6 containing an anionic surfactant 7 (with its counterion 8). FIG. 3 depicts a hypothetical method, or “thought experiment”, which is intended to illustrate a fundamental difference between the current invention and a simple mixing of surfactant and drug. In the latter method, which is the method of Lynch and Spicer cited elsewhere herein, one can form a material which contains the surfactant (or “anchor”) in the liquid crystal (or other nanostructured phase), and this matrix can later accept the drug—that is, the drug is added in its usual form to a thermodynamically stable material containing the surfactant with counterion intact. However, in the current invention, in the case where a drug is ion-paired to an ionic surfactant moiety, namely surfactant minus counterion, then as in FIG. 3, this would require the preparation of a matrix containing the charged, counterion-free surfactant moiety—which violates charge neutrality and thus fundamental thermodynamic laws—and later adding the drug also in the form of a charged, counterion-free drug moiety, which is also thermodynamically prohibited. The scenario of forming a complex in situ inside a cubic phase or similar material is in most cases absurd as well. Such complexes require intelligent application of synthetic chemistry procedures (including, for example, the use of organic solvents that are not pharmaceutically-acceptable).

It should be noted that, in the terminology of this patent, the formation of a coordinate bond between two moieties results in a complex, and this differs in a number of respects from the formation of a salt, or ion pair. Coordinate bonds are typically formed by transition metals, in which the metallic compound serves as a Lewis acid. The second moiety in such a case is a Lewis base, and the metal donates both electrons that make up the bond between the Lewis base and Lewis acid in the coordination compound. Such compounds are often colored, and require somewhat more intricate and careful chemistry than the production of salts; for example, oxidation states can change, polymers can form, and reactants can complex with organic solvents. Dimethylacetamide is a particularly useful solvent for such reactions for a number of reasons: it has less of a tendency to complex than, for example, DMSO; it can be vacuumed off; and it is of low toxicity. Silver nitrate is a useful reagent for the removal of chloride or bromide from pre-existing complexes, and the resulting nitrate group is generally easily displaced.

Anionic materials. For formulations intended for administration by injection or other non-oral routes, especially preferred anionic moieties for binding the drug are: docusate, dodecylsulfate, deoxycholic acid (and related cholates), stearic acid and other 18-carbon fatty acids including oleic, linoleic, and linolenic acids, gentisic acid, hydrophobic amino acids including tryptophan, tyrosine, leucine, isoleucine, aspartic acid, cystine, and their N-methylated derivatives, particularly N-acetyltryptophan, myristyl gamma-picolinium chloride, phosphatidylserine, phosphatidylinositol, phosphatidylglycerol (particularly dimyristoyl phosphatidylglycerol), and other anionic and acidic phospholipids. The person with skill in the art will recognize docusate as the anionic moiety of the surfactant docusate sodium (also known as Aerosol OT), and dodecylsulfate as the anionic moiety of the surfactant sodium dodecylsulfate, or SDS. Surface-active polypeptides and proteins, such as casein and albumin, may also be used, though their high molecular weights dictate a large protein:drug weight ratio, meaning that the molar amount of drug that will be bound by such an approach will be very small.

For formulations intended for oral administration, the above anionic compounds can be used, but in addition there are a number of other compounds that can provide the anion. These include ascorbyl palmitate, stearoyl lactylate, glycyrrhizin, monoglyceride citrate, stearyl citrate, sodium stearyl fumarate, JBR-99 rhamnolipid (and other biosurfactants from Jeneil Biosurfactant), glycocholic acid, taurocholic acid, and taurochenodeoxycholic acid.

Especially preferred anionic surfactants are: sodium oleate, sodium dodecyl sulfate, sodium diethylhexyl sulfosuccinate, sodium dimethylhexyl sulfosuccinate, sodium di-2-ethylacetate, sodium 2-ethylhexyl sulfate, sodium undecane-3-sulfate, sodium ethylphenylundecanoate, carboxylate soaps of the form ICₙ, where the chain length n is between 8 and 20 and I is a monovalent counterion such as sodium, potassium, ammonium, etc.,

Surfactants and lipids. In addition to the charged bilayer-associated moiety, it is normal in the practice of the current invention to incorporate other surfactants and lipids, and in fact a good methodology is to use a mixture of surfactants, one of which is effective at forming reversed hexagonal, or especially reversed cubic, phases in equilibrium with water (that is, insoluble, or “non-erodable” phases), and the other comprises the moiety that can bind the drug of interest. Preferred surfactants which are FDA-approved as injectables include benzalkonium chloride, sodium deoxycholate, myristyl-gamma-picolinium chloride, Poloxamer 188, polyoxyl castor oil and related PEGylated castor oil derivatives such as Cremophor EL, Arlatone G, sorbitan monopalmitate, Pluronic 123, and sodium 2-ethylhexanoic acid. Other low-toxicity surfactants and lipids, which are of at least relatively low solubility in water, that are preferred for the present invention for products intended for a number of routes of administration, include: acetylated monoglycerides, aluminum monostearate, ascorbyl palmitate free acid and divalent salts, calcium stearoyl lactylate, ceteth-2, choleth, deoxycholic acid and divalent salts, docusate calcium, glyceryl stearate, stearamidoethyl diethylamine, ammoniated glycyrrhizin, lanolin nonionic derivatives, magnesium stearate, methyl gluceth-120 dioleate, monoglyceride citrate, octoxynol-1, oleth-2, oleth-5, peg vegetable oil, peglicol-5-oleate, pegoxol 7 stearate, poloxamer 331, polyglyceryl-10 tetralinoleate, polyoxyethylene fatty acid esters, polyoxyl castor oil, polyoxyl distearate, polyoxyl glyceryl stearate, polyoxyl lanolin, polyoxyl-8 stearate, polyoxyl 150 distearate, polyoxyl 2 stearate, polyoxyl 35 castor oil, polyoxyl 8 stearate, polyoxyl60 castor oil, polyoxyl 75 lanolin, polysorbate 85, sodium stearoyl lactylate, sorbitan sesquioleate, sorbitan trioleate, stear-o-wet c, stear-o-wet m, stearalkonium chloride, stearamidoethyl diethylamine, steareth-2, steareth-10, stearic acid, stearyl citrate, sodium stearyl fumarate or divalent salt, trideceth 10, trilaneth-4 phosphate, lipoic acid, Detaine PB, JBR-99 rhamnolipid (from Jeneil Biosurfactant), glycocholic acid and its salts, taurochenodeoxycholic acid (particularly combined with vitamin E), tocopheryl phosphonate, tocopheryl peg 1000 succinate Cholesterol, vaxfectin, cardiolipin, dodecyl-N,N-dimethylglycine, and lung surfactant (Exosurf, Survanta).

The current inventor has found the following pharmaceutically-acceptable surfactants to be particularly useful in forming insoluble reversed cubic and hexagonal phases capable of incorporating ion-pairing constituents: phosphatidylcholine, phosphatidylethanolamine, Arlatone G, Tween 85, glycerol monooleate and other long-chain unsaturated monoglycerides, sorbitan monooleate, zinc and (to a lesser extent) calcium docusate, and Pluronics with less than about 30% PEO groups by weight, especially Pluronic L122 and to a lesser extent L101; Pluronic P123 also forms reversed cubic and hexagonal phases but has a significant solubility in water which can limit its usefulness. The low-MW ethoxylated surfactants OE-2 and OE-5 (oleyl alcohol ether-linked to either 5 or 2 PEG groups) are useful in this respect but their approval in drug formulations is limited, depending on the route of administration.

Cationic surfactants. As discussed herein, currently the selection of pharmaceutically-acceptable cationic surfactants is primarily limited to myristyl-gamma-picolinium chloride and benzalkonium chloride. However, a number of other cationic lipids and surfactants are currently under investigation as pharmaceutical excipients, including: tocopheryl dimethylaminoacetate hydrochloride, cytofectin gs, 1,2-dioleoyl-sn-glycero-3-trimethylammonium-propane, cholesterol linked to lysinamide or omithinamide, dimethyldioctadecyl ammonium bromide, 1,2-dioleoyl-sn-3-ethylphosphocholine and other double-chained lipids with a cationic charge carried by a phosphorus or arsenic atom, trimethyl aminoethane carbamoyl cholesterol iodide, O,O′-ditetradecanoyl-N-(alpha-trimethyl ammonioacetyl) diethanolamine chloride (DC-6-14), N-[(1-(2,3-dioleyloxy)propyl)]-N—N—N-trimethylammonium chloride, N-methyl-4-(dioleyl)methylpyridinium chloride (“saint-2”), lipidic glycosides with amino alkyl pendent groups, 1,2-dimyristyloxypropyl-3-dimethylhydroxyethyl ammonium bromide, bis[2-(1-phenoxyundecanoate)ethyl]-dimethylammonium bromide, N-hexadecyl-N-10-[O-(4-acetoxy)-phenylundecanoate]ethyl-dimethylammonium bromide, 3-beta-[N—(N′,N′-dimethylaminoethane)-carbamoyl.

Other useful bilayer-associated compounds. Other suitable membrane-associated amphiphiles for use in the instant invention, which can take up a charge under at least some conditions, include: fatty acids, phenolic compounds such as eugenol, isoeugenol, quinolines, hydroxyquinolines and benzoquinolines, tricyclics such as carbazole, phenothiazine, etc., pigments, chlorophyll, certain natural oil extracts particularly those which are phenolic (such as clove oil, ginger oil, basil oil), biosurfactants (such as Jeneil's “JBR-99”). One can imagine using amphiphilic proteins and polypeptides including gramicidin, casein, albumin, glycoproteins, lipid-anchored proteins, receptor proteins and other membrane proteins such as proteinase A, amyloglucosidase, enkephalinase, dipeptidyl peptidase IV, gamma-glutamyl transferase, galactosidase, neuraminidase, alpha-mannosidase, cholinesterase, arylamidase, surfactin, ferrochelatase, spiralin, penicillin-binding proteins, microsomal glycotransferases, kinases, bacterial outer membrane proteins, and histocompatibility antigens. As is well known, every protein has a net charge except at its isoelectric point, and thus a pharmaceutically-acceptable membrane-associated protein is suitable for use in the present invention as long as the pH is away from its isoelectric point. A few such proteins are currently accepted as inactive ingredients for pharmaceutical preparations, at least under some conditions, and these include gluten, casein, and albumin. However, as pointed out elsewhere herein, the molar amounts of such high-MW compounds that can be incorporated are of course small, simply by virtue of their MW, and since the net charge (relating to the number of drug molecules that can be bound) is usually small, the drug loading as a weight fraction of the matrix is very limited. Since most pharmaceutical actives have molecular weights less than about 1,000 it follows that the preferred molecular weight of the bilayer-associated moiety should preferably be less than about 5,000 (the generally accepted cutoff between polymers and oligomers or small molecules), and preferably less than about 1,000.

One limitation of the method is encountered due to the fact that there are no primary amines with high octanol-water partition coefficients that are approved for oral or injectable drug formulations, except for zwitterionic or amphoteric compounds such as amino acids in which the amino group is already ion-paired. Thus, outside of benzalkonium chloride and myristyl-gamma-picolinium chloride, it is difficult to reliably bind anionic drugs in a way that does not require tuning of pH specifically for that binding; for some drugs which must be formulated in a certain pH range, this must be considered by the formulator. Example 5 herein gives an example of the use of arginine for the binding of alendronate, and it should be noted that the approach calls for the conversion of sodium alendronate-the usual marketed form of alendronate—to the free acid, before binding it to the arginine.

Pharmaceutical Actives for the Present Invention.

Platinum drugs. Platinum compounds that can be formulated using this approach include, but are not limited to: Carboplatin, CI-973, Cisplatin, Enloplatin, Iproplatin, JM216, L-NDDP, Lobaplatin, Oxaliplatin, Spiroplatin, Tetraplatin, Zeniplatin, AMD-473, BBR-3464, Transplatin, Thioplatin, ZD0473, Satraplatin, AR-726, SPI-077, Lipoplatin, Intradose-CDDP, Nedaplatin, AP5070, Atrigel, and other mononuclear and multinuclear platinum compounds. Multinuclear compounds can benefit considerably from this invention, since the binding of thiols to such compounds, which is inhibited by the complexes of this invention, can have disastrous effects: the binding of thiols by displacement of chlorides can break apart the bridges between platinum atoms and release highly toxic residues with long-lasting side effects.

Other anticancer drugs. In view of the demanding requirements for the delivery of pharmaceuticals in the treatment of cancers, the advantages and flexibility of the present invention make it particularly attractive in the delivery and release of antineoplastic agents, such as for example, the following: Alkylating Agents; Aziriainessuch asBenzodepa, Carboquone, Meturedepa, Uredepa; Ethyleneimines and Methylmelamines such as Altretamine, Triethylenemelamine, Triethylenephosphoramide, Triethylenethiophosphorami- de, Trimethylolmelamine; Nitrogen Mustards such as Chlorambucil, Chloramphazine, Cyclophosphamide, Estramustine, Ifosfamide, Mechlorethamine, Mechlorethamine Oxide Hydrochloride, Melphalan, Novembichin, Phenesterine, Prednimustine, Trofosfamide, Uracil, Mustard; Carmustine, Chlorozotocin, Fotemustine, Lomustine, Nimustine, Ranimustine; Antibiotic antineoplastics such as Actinomycin FI, Anthramycin, Azaserine, Bleomycins, Actinomycin, Carubicin, Carzinophilin, Chromomycins, Dactinomycin, Daunorubicin, 6-Diazo-5-OXO-Leucine, Doxorubicin, Epirubicin, Mitomycins, Mycophenolic Acid, Nogalamycin, Olivomycins, Peplomycin, Plicarmcin, Porfiromycin, Puromycin, Streptonigrin, Streptozocin, Tubercidin, Ubenimex, Zinostatin, Zorubicin; Antimetabolites; Folic Acid Analogs such as Denopterin, Methotrexate, Pteropterin, Trimetrexate; PurineAnalogs such as Fludarabine, 6-Mercaptopurine, Thiamiprine, Thioguanine; Pyrimidine Analogs such as Ancitabine, Azacitidine, 6-Azauridine, Carmofur, Cytarabine, Doxifluridine, Enocitabine, Floxuridine, Fluorouracil, Tegafur; Aceglatone, Amsacrine, Bestrabucil, Bisantrene, Carboplatin, Cisplatin, Defosfamnide, Demecolcine, Diaziquone, Eflorithine, Elliptinium Acetate, Etoglucid, Interferon-alpha, Interferon-beta, Interferon-gamma, Interleukin-2, Lentinan, Lonidamine, Mitoguazone, Mitoxantrone, Mopidamol, Nitracrine, Pentostatin, Phenamet, Pirarubicin, Podophyllinic Acid, 2-Ethylhydrazide, Procarbazine, PSK09, Razoxane, Sizofiran, Spirogermanium, Taxol, Tenuazonic Acid, Triaziquone, 2,2′,2,1,1-Trichlorotriethylami- ne, Urethan, Vinblastine, Vincristine, Vindesine; Antiadrenals such as Aminoglutethimide, Mitotane, Trilostane; Antiestrogens such as Tamoxifen, Toremifene; Estrogens such as Polyestradiol Phosphate; LH-RH Analogs such as Buserelin, Goserelin, Leuprolide, Triptorelin; Antineoplastic Adjuncts; Folic Acid Replenishers such as Folinic Acid; Uroprotectives such as Mesna; and others, such as Dacarbazine, Mannomustine, Mitobronitol, Mitolactol, and Pipobroman.

Other charged drugs. Other pharmaceutical compounds that are particularly well-suited for the instant invention, and thus have a net charge over certain ranges of pH, and also suffer from problems or limitations in the currently-marketed formulations, include: Dacarbazine, Ifosfamide, Streptozocin, Thiotepa, Nandrolone decanoate, Fentanyl citrate, Albendazole, Esmolol, Bleomycin, Dactinomycin, Amikacin, Gentamicin, Netilmicin, Streptomycin, Tobramycin, Doxorubicin, Epirubicin, Idarubicin, Valrubicin, Bacitracin, Colistimethate, Oxybutinin, Antithrombin III Human, Heparin, Lepirudin, Adenosine phosphate, Amphotericin B, Enalaprilat, Cladribine, Cytarabine, Fludarabine phosphate, Gemcitabine, Pentostatin, Vinblastine, Vincristine, Vinorelbine, Batimastat, Rituximab, Trastazumab, Abciximab, Eptifibatide, Tirofiban, Droperidol, Aurothioglucose, Capreomycin disulfide, Acyclovir, Cidofovir, Pentafuside, Saquinavir, Ganciclovir, Cromolyn, Aldesleukin, Denileukin, Edrophonium, Infliximab, Doxapram, Irinotecan, Hemin, Daunorubicin, Teniposide, Trimetrexate, Octreotride, Ganirelix acetate, Histrelin acetate, Somatropin, Epoetin, Filgrastim, Oprelvekin, Leuprolide, Basiliximab, Daclizumab, Glatiramer acetate, Interferons, Muromonab-CD3, Clyclosporin A, Milrinone lactate, Buprenorphine, Nalbuphine, Urofollitropin, Desmopressin, Carboplatin, Cisplatin, Mitoxantrone, Estradiol, Hydroxyprogesterone, L-Thyroxine, Etanercept, Neostigmine, Epoprostenol, Methoxamine, Midazolam, Bupivacaine and other local anesthetics of this class (commonly referred to as “caines”), Heparin, Insulin, Antisense compounds, Ketoprofen, Alendronate, Etidronate, Zoledronate, Ibandronate, Risedronate, and Pamidronate. These compounds represent the following classes of drug: Alkylating agent, Anabolic steroid, Analgesic, Androgen, Anthelmintic, Antiadrenergic, Antibiotic, Antibiotic, aminoglycoside, Antibiotic, antineoplastic, Antibiotic, polypeptide, Anticholinergic, Anticoagulant, Anticonvulsant, Antifungal, Antihypertensive, Antimetabolite, Antimitotic, Antineoplastic, Antiplatelet, Antipsychotic, Anesthetic, Antirheumatic, Antituberculosal, Antiviral, Antiviral (HIV), Asthma anti-inflammatory, Biological response modifier, Cholinergic muscle stimulant, CNS stimulant, DNA topoisomerase inhibitor, Enzyme inhibitor, Epipodophyllotoxin, Folate antagonist, Gastric antisecretory, Gene therapy agents, Gonadotropin-releasing, Growth hormone, Hematopoietic, Hormone, Immunologic agent, Immunosuppressant, Inotropic agent, Local anesthetic, Narcotic agonist/antagonist, Ovulation stimulant, Pituitary hormone, Platinum complex, Sex hormone, Thyroid hormone, TNF inhibitor (arthritis), Urinary cholinergic, Vasodilator, and Vasopressor. We note that the current invention is also very well suited for the incorporation of functional excipients that, for example, improve absorption of poorly-absorbed drugs, in some cases by inhibiting drug efflux proteins. As discussed in more detail elsewhere herein, there are a number of sites within, and at the surface of the particles, where actives, excipients, and functional excipients can be localized within the context of this invention.

Routes of Administration. The compositions of the present invention may be administered by any of a variety of means which are well known to those of skill in the art. These means include but are not limited to oral (e.g. via pills, tablets, lozenges, capsules, troches, syrups and suspensions, and the like) and non-oral routes (e.g. parenterally, intravenously, intraocularly, transdermally, via inhalation, and the like). The compositions of the present invention are particularly suited for internal (i.e. non-topical) administration. The present invention is especially useful in applications where a difficultly soluble pharmaceutical active is to be delivered internally (i.e. non-topical), including orally and parenterally, wherein said active is to be miscible with a water continuous medium such as serum, urine, blood, mucus, saliva, extracellular fluid, etc. In particular, an important useful aspect of many of the structured fluids of focus herein is that they lend themselves to formulation as water continuous vehicles, typically of low viscosity.

### EXAMPLES

### Example 1

Cisplatin, in the amount 7.6 mg, was dissolved in 1.50 gm of dimethylacetamide, and 0.20 gm of the acidic-rich (phosphatidylinositol-rich) phospholipid mixture “Epikuron 105” (Lucas-Meyer) was added and mixed thoroughly. A control sample was prepared with the same amounts but with the cisplatin omitted. Phosphorus (³¹P) NMR was then run on both the sample and the control. Several drops of D₂O were added to aid in the locking of the NMR signal.

The resulting NMR spectra showed a systematic shift of 6 peaks, indicating the formation of a complex between phospholipid (predominantly phosphatidylinositol) and cisplatin (or more accurately, the cationic compound formed by the displacement of chloride ions from cisplatin). The positions of the six ³¹P NMR peaks (in ppm) in the sample and control are listed in the table below.

| | | | | | | |
|---|---|---|---|---|---|---|
| Peak position for sample | 0.092 | 0.795 | 1.041 | 1.374 | 1.609 | 2.015 |
| Peak position for control | 0.191 | 0.906 | 1.214 | 1.522 | 1.793 | 2.175 |

The systematic downfield shift is due to the change in local chemical environment at the phosphorus atom due to the complexation with the platinum compound. As is well known in the art, this sort of complexation with platinum generally causes a downfield shift, due to the high electron density associated with the platinum atom.

### Example 2

Cisplatin, in the amount 7.6 mg, was dissolved in 1.5 gm dimethylacetamide together with 0.20 gm of Epikuron 105. Cisplatin, 8 mg, was then dissolved in 0.5 gm of dimethylacetamide, to make a control sample. Platinum nuclei were investigated, using ¹⁹⁵Pt NMR, with several drops of D₂O added. The peak position shifted from −2112 ppm for the control to −2090 ppm for the phospholipid-containing sample. Again, this shift, which is significant, is due to complexation of the platinum compound with the phosphorus compound (lipid). The presence of the phosphorus atom in the vicinity of the platinum atom results in a higher local electron density in the neighborhood of the platinum atom, causing the shift to move downfield as compared to the shift (−2112) in dimethylacetamide (which is lacking in heavier atoms).

### Example 3

A phosphatidylcholine-rich lecithin, Epikuron 200 (Lucas-Meyer), in the amount 0.371 gm, was combined with 0.679 gm of the acidic-lipid-rich phospholipid mixture Epikuron 105, and 0.251 gm of essential oil of ginger, 0.283 gm of water, and 0.004 gm of potassium hydroxide, to form a reversed cubic phase. To this cubic phase 25.4 mg of cisplatin was added, and 0.70 gm of dimethylacetamide was then added to help solubilize the cisplatin, the entire mixture being stirred thoroughly. Following this, the mixture was stirred into about 1.5 gm of water, which resulted in the dispersing of a significant portion of the lipid-rich phase into the water.

¹⁹⁵Pt NMR was then performed on the sample, yielding a single peak with a chemical shift of −2090 ppm. This matches the shift seen in Example 2 for the platinum compound complexed to phospholipid, indicating that the cisplatin (minus chloride) is complexed to the anionic phospholipid. The high degree of lipophilicity of the complex, which follows from the highly lipophilic character of the phospholipid (which contains two acyl chains of carbon length 16 or 18, predominantly, on each molecule) means that the complex is clearly partitioned into the particles of the lipid-rich, reversed cubic phase.

### Example 4

In this experiment, the silver nitrate-based method described above was used to produce a docusate-drug complex. The experiment started with a dinuclear platinum compound, with an average of 1.25 chloride ions per molecule, and a bridge between the two platinum atoms that was based on a spermidine derivative. An amount 14.8 mg of this compound was dissolved in 1.6 gm of methanol, and this was combined with a solution of 10.7 mg silver nitrate in 1.0 gm of methanol, with a slight heating applied to aid dissolution. Silver chloride then precipitated, indicating that the chloride ions from the platinum had been displaced by nitrate ions. A second solution was prepared with 22.7 mg of sodium docusate all dissolved in 0.4 gm of methanol. In order to precipitate the sodium nitrate elimination product, 3 gms of tetrahydrofuran were added, and the methanol evaporated under nitrogen, yielding a precipitate, which was centrifuged out. To the THF solution of the product were added 0.44 gm of sodium docusate (to give a 3-fold excess), and the THF dried off. Of the resulting docusate-platinum drug complex, 25 mg were combined with 125 mg of glycerol monooleate and 100 mg of water, and stirred vigorously. The result was a perfectly transparent, optically isotropic, high viscosity cubic phase in which the platinum compound (with the chloride-to-docusate substitutions) was solubilized. Examination in a phase contrast microscope with a 40× objective (400× overall magnification) did not reveal any undissolved material, consistent with the optical isotropy. Strong evidence of the complexation of the platinum compound is afforded by the fact that the glycerol monooleate—water cubic phase was unable to dissolve the original platinum compound even at the low level of 2 mg drug per gm of cubic phase; the loading achieved with the docusate complexation is equivalent to 18 mg drug per gram of cubic phase, with full solubilization of this amount of drug.

### Example 5

This Example reports a composition in which the anionic drug alendronate, after conversion to its free acid form by reaction with hydrochloric acid, was ion-paired with the cationic amino acid arginine.

The antiosteolytic drug Alendronate (as the free acid) was incorporated into a cubic phase based on the ethoxylated, hydrogenated castor oil surfactant Arlatone G (from Uniquema). Alendronate free acid (0.087 grams) was solubilized in a mixture of 0.479 grams of essential oil of ginger, 0.052 grams of arginine, 0.439 grams of water, and 0.940 grams of Arlatone G. When this cubic phase, which exists in equilibrium with water, was overlain with a large excess of water and allowed to incubate together with the excess water for two days, it was found that the amount of alendronate which leaked out of the cubic phase into the water was so small as to be undetectable.

An amount 0.995 grams of this cubic phase were placed in a test tube and 2.509 grams of hydrogenated cottonseed oil added, and the entire contents were heated to 90° C. to melt the oil. The sample was immediately sonicated in a hot water bath with vigorous shaking every 30 seconds, for 3 minutes. The test tube was then placed in an ice bath to solidify the oil with particles dispersed throughout the trigylceride. The resulting solid was then milled by the application of mechanical energy to an average particle size of several hundred microns; further reduction in size can readily be accomplished by milling methods well known in the art. SAXS analysis of this sample was incomplete but clearly showed the presence of a Bragg peak at approximately 10.9 nm which was due to long-range order in the liquid crystalline particle interior, in addition to peaks at 4.51, 2.26, and 1.52 nm due to the lattice of the frozen triglyceride. The existence of the peak at 10.9 nm was confirmed by analysis of the X-ray spectrum using the peak-analysis program JADE. This material is suitable for use in the oral delivery of the drug alendronate, which currently suffers from very poor availability as the orally administered drug Fosamax.

I claim:

1. A pharmaceutical which is composed of an association complex between two moieties,
wherein a first of said two moieties is pharmaceutically active, and is larger than a single element in size,
wherein a second of said two moieties consists essentially of one or more compounds which respectively form, when combined with a cationic or anionic counterion, either forms
(i) a pharmaceutically acceptable anionic surfactant or a pharmaceutically acceptable cationic surfactant, or
(ii) a pharmaceutically acceptable salt that has an octanol-water partition coefficient that is greater than about 100, and
wherein said pharmaceutical is solubilized in one of a reversed cubic phase, a reversed hexagonal phase, or an L3 phase.

2. The pharmaceutical of claim 1 wherein said pharmaceutical is physically present in a reversed cubic phase.

3. The pharmaceutical of claim 1 wherein said pharmaceutical is physically present in a reversed hexagonal phase.

4. The pharmaceutical of claim 1 wherein said pharmaceutical is physically present in an L3 phase.

5. The pharmaceutical of claim 1 wherein said second of said two moieties, when combined with a cationic or anionic counterion forms (i) a pharmaceutically acceptable anionic surfactant or pharmaceutically acceptable cationic surfactant.

6. The pharmaceutical of claim 5 wherein said second of said two moieties, when combined with a cationic counterion forms an anionic surfactant.

7. The pharmaceutical of claim 5 wherein said second of said two moieties, when combined with an anionic counterion forms a cationic surfactant.

8. The pharmaceutical of claim 1 wherein said second of said two moieties, when combined with a cationic or anionic counterion forms (ii) a pharmaceutically acceptable salt that has an octanol-water partition coefficient of at least 100.

9. The pharmaceutical of claim 8 wherein said second of said two moieties, when combined with a cationic counterion forms a pharmaceutically acceptable salt that has an octanol-water partition coefficient of at least 100.

10. The pharmaceutical of claim 8 wherein said second of said two moieties, when combined with an anionic counterion forms a pharmaceutically acceptable salt that has an octanol-water partition coefficient of at least 100.

11. The pharmaceutical of claim 1 wherein said second of said two moieties, when combined with a cationic or anionic counterion forms (ii) a pharmaceutically acceptable salt that has an octanol-water partition coefficient of at least 1000.

12. The pharmaceutical of claim 1 wherein said pharmaceutical is present as a particle.

13. The pharmaceutical of claim 12 further comprising a coating on said particle.

14. The pharmaceutical of claim 13 wherein said coating has lamellar domains.

15. The pharmaceutical of claim 13 wherein said coating has nonlamellar domains.

16. The pharmaceutical of claim 15 wherein at least some of said nonlamellar domains are crystalline.

17. The pharmaceutical of claim 13 wherein said coating has amorphous domains.

18. The pharmaceutical of claim 1 wherein said pharmaceutical is present as a dispersion of particles in a carrier.

19. The pharmaceutical of claim 1 wherein said pharmaceutical is present as a dispersion of particles in a matrix.

20. The pharmaceutical of claim 1 wherein said first of said two moieties includes at least one platinum atom.

21. The pharmaceutical of claim 1 wherein said first of said two moieties is a cationic form of a pharmaceutically active which lacks a halogen atom, and wherein said second of said two moieties is an anion.

22. The pharmaceutical of claim 21 wherein said anion includes a hydrophobic portion.

23. The pharmaceutical of claim 1 wherein said second of said two moieties is a lipid.

24. The pharmaceutical of claim 1 wherein said association complex of said two moieties is electrostatic.

25. The pharmaceutical of claim 1 wherein said association complex of said two moieties includes a coordinate bond.

26. The pharmaceutical of claim 1 wherein said association complex of said two moieties includes an ionic bond.

27. The pharmaceutical of claim 1 wherein said first of said two moieties is selected from the group consisting of Carboplatin, CI-973, Cisplatin, Enloplatin, Iproplatin, JM216, L-NDDP, Lobaplatin, Oxaliplatin, Spiroplatin, Tetraplatin, Zeniplatin, AMD-473, BBR-3464, Transplatin, Thioplatin, ZD0473, Satraplatin, AR-726, SPI-077, Lipoplatin, Intradose-CDDP, Nedaplatin, AP5070, Atrigel, and other mononuclear and multinuclear platinum compounds.

28. The pharmaceutical of claim 1 wherein said first of said two moieties is selected from the group consisting of antineoplastic agents, Ethyleneimines and Methvlmelamines, Nitrogen Mustards, Carmustine, Chlorozotocin, Fotemustine, Lomustine, Nimustine, Ranimustine, Antibiotic antineoplastics, Folic Acid Analogs, PurineAnalogs, Pyrimidine Analogs, Antiadrenals, Antiestrogens, Estrogens, LH-RH Analogs, Antineoplastic Adjuncts, Folic Acid Replenishers, Uroprotectives, Dacarbazine, Mannomustine, Mitobronitol, Mitolactol, and Pipobroman.

29. The pharmaceutical of claim 1 wherein said second of said two moieties is selected from the group consisting of benzalkonium chloride, sodium deoxycholate, myristyl-gamma-picolinium chloride, Poloxamer 188, polyoxyl castor oil and related PEGylated castor oil derivatives, acetylated monoglycerides, aluminum monostearate, ascorbyl palmitate free acid and divalent salts, calcium stearoyl lactylate, ceteth-2, choleth, deoxycholic acid and divalent salts, docusate calcium, glyceryl stearate, stearamidoethyl diethylamine, amumoniated glycyrrhizin, lanolin nonionic derivatives, magnesium stearate, methyl gluceth-120 dioleate, monoglyceride citrate, octoxynol- 1, oleth-2, oleth-5, peg vegetable oil, peglicol-5-oleate, pegoxol 7 stearate, poloxamer 331, polyglyceryl-10 tetralinoleate, polyoxyethylene fatty acid esters, polyoxyl castor oil, polyoxyl distearate, polyoxyl glyceryl stearate, polyoxyl lanolin, polyoxyl-8 stearate, polyoxyl 150 distearate, polyoxyl 2 stearate, polyoxyl 35 castor oil, polyoxyl 8 stearate, polyoxyl60 castor oil, polyoxyl 75 lanolin, polysorbate 85, sodium stearoyl lactylate, sorbitan sesquioleate, sorbitan trioleate, stear-o-wet c, stear-o-wet m, stearalkonium chloride, stearamidoethyl diethylamine, steareth-2, steareth-10, stearic acid, stearyl citrate, sodium stearyl fumarate or divalent salt, trideceth 10, trilaneth-4 phosphate, lipoic acid, Detaine PB, JBR-99 rhamnolipid (from Jeneil Biosurfactant), glycocholic acid and its salts, taurochenodeoxycholic acid (particularly combined with vitamin E), tocopheryl phosphonate, tocopheryl peg 1000 succinate Cholesterol, vaxfectin, cardiolipin, dodecyl-N,N-dimethylglycine, lung surfactants, phosphatidylcholine, phosphatidylethanolamine, Arlatone G, Tween 85, glycerol monooleate and other long-chain unsaturated monoglycerides, sorbitan monooleate, zinc and calcium docusate, and Pluronics with less than about 30% PEO groups by weight, and low-MW ethoxylated surfactants.

30. A method of delivering a pharmaceutical to a patient, comprising
administering to said patient a pharmaceutical which is composed of an association complex between two moieties,
wherein a first of said two moieties is pharmaceutically active, and is larger than a single element in size,
wherein a second of said two moieties consists essentially of one or more compounds which respectively form, when combined with a cationic or anionic counterion, either forms
(i) a pharmaceutically acceptable anionic surfactant or a pharmaceutically acceptable cationic surfactant, or
(ii) a pharmaceutically acceptable salt that has an octanol-water partition coefficient that is greater than about 100, and
wherein said pharmaceutical is solubilized in one of a reversed cubic phase, a reversed hexagonal phase, or an L3 phase.

31. The method of claim 30 wherein said step of administering is performed by oral route.

32. The method of claim 30 wherein said step of administering is performed by injection.
